(19) United States
(12) Patent Application Publication
(10) Pub. No.: US 2004/0176697 A1
(43) Pub. Date: Sep. 9, 2004

(54) Methods of analyzing atrial fibrillations

(76) Inventors: Lukas Kappenberger, Lausanne (CH); Jean-Marc Vesin, Thonon (FR)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/700,999

(22) Filed: Nov. 3, 2003

Related U.S. Application Data
(60) Provisional application No. 60/423,040, filed on Nov. 1, 2002.

(51) Int. Cl. A61B 5/046

(52) U.S. Cl. 600/518

(57) ABSTRACT

The present invention relates generally to analysis of electrocardiograms (ECGs) during atrial fibrillation. In particular, the invention relates to the use of such methods for the the creation and validation of cardiac models and use in the refined diagnosis of heart disease.

### RELATED APPLICATIONS

This application claims the benefit of U.S.S.No. 60/423,040, filed Nov. 1, 2002, which is incorporated herein by reference in its entirety.

### FIELD OF THE INVENTION

The present invention relates generally to analysis of electrocardiograms (ECGs) during atrial fibrillation and to methods for the creation and use of cardiac models to diagnose heart disease.

### BACKGROUND OF THE INVENTION

Atrial fibrillation (AF) is an uncoordinated electrical activation of the atria, classified as supraventricular arrhythmia. The disordered depolarization of the atrium leads to an inefficient atrial contraction and, thus, to impaired cardiac function. On an electrocardiogram, the P wave is replaced by fibrillation (F) waves varying in shape, size and timing with rates over 350/min. Those findings are associated with an irregular ventricular response, which is often rapid in case of physiologic atrio-ventricular conduction.

Atrial fibrillation is the most common arrhythmia in clinical practice and it is responsible of about one third of hospitalizations for arrhythmia problems. The prevalence of AF is 0.4% of the general population. AF is more frequent in the elderly, as its prevalence doubles with each advancing decade of age, from 0.5% at age 50-59 years to almost 9% at age 80-89 years. Furthermore, with increasing life expectancy, the prevalence is expected to double in the next fifty years. The yearly incidence of AF, also related to advancing age, is less than 0.1% under 40 years and over 1.5% above 80 years. Atrial flutter is similar to atrial fibrillation in many ways but is less common. The two arrhythmias often occur together in the same patient and in the past, have often been indistinguishable by ECG.

In the early twentieth century, two potential mechanisms arose regarding the electrical mechanism of AF: circuits of reentry (known as multiple wavelets hypothesis) and automaticity in one or several ectopic foci. Later, Moe et al. advanced the hypothesis that AF was sustained by multiple re-entrant circuits with spatial and temporal variability that act like depolarization-front wavelets on both atria. Allessie et al. provided the first experimental evidence supporting this hypothesis when mapping the electrical atrial activity during AF. Over the past fifty years this theory has been the predominant concept of AF. However, recent observations suggest the existence of rapidly firing-atrial foci, most often found in pulmonary veins. Thus, it is commonly felt that both mechanisms are involved in AF, either alternatively, or in combination.

Atrial fibrillation's rhythm has been described as totally disorganized, but there is growing evidence of some structure in its activation. Many methods of quantification of organization have been proposed, such as single-site recordings, two simultaneous recordings and multiple recordings (i.e. mapping). Single-site recordings measure temporal organization, while multiple-site recording provides for the assessment of spatial relationships.

A shortcoming of these recordings is that they are obtained from invasive measures, and a determination regarding whether the cardiac organization could be quantified from a surface ECG has not been made prior to the present invention. Moreover, the ability to classify AF according to ECG appearances and relate the classification to distinct underlying pathologies was therefore not possible.

Only a few references in the relevant literature deal directly with the problem of AF classification, and those that do so study AF classification with respect to AF manifestation, not substrate. Many studies have focused on abnormal cardiac rhythm detection (on the basis of earlier works on ventricular tachycardia and fibrillation). Atrial fibrillation is often diagnosed, but not measured or classified by electrocardiogram (ECG or EKG). One limitation in the current use of ECG to classify atrial fibrillation is that such classification is not based upon the underlying atrial disorders, such as dilated cardiomyopathy, hypertrophic cardiomyopathy, rheumatismal valvular disease, pericarditis, or focal atrial fibrillation, but instead is based only on clinical observation, primarily the patient's history, taking into account only mode of appearance, persistence, and/or duration. Detailed analysis of the atrial fibrillatory ECG has not been performed or described in the art.

ECGs have recently received some attention for potential use in AF analysis, due in part to their (relatively) easier tractability in terms of deformation and signal-to-noise ratio. ECG can be used to discriminate between AF, sinus rhythm, atrial flutter, and supraventricular tachycardia using a specific catheter with orthogonally placed electrodes. Wavelet transform potential for cardiac rhythm classification has also been studied using ECGs. The spatial or temporal structure of AF electrograms has been examined from the viewpoint of synchronization, linear and nonlinear autoregressive model properties. Also, several ad hoc and statistical parameters (such as entropy) have been compared in their capacity to discriminate AF classes. Additional studies have begun to examine methods to quantify the spatio-temporal organization of AF; one study has proposed to examine the space constant of the approximately exponential decay of correlation with electrode distance. Further, AF organization has been quantified by the mean-squared error in the linear prediction between pairs of electrograms, and in 12 non-linear coupling (correlation dimension and correlation entropy).

Potential problems regarding the use of ECG in classifying AF include the task of separating atrial and ventricular activities. Early studies proposed an average beat substraction approach to QRST complex cancellation, and an adaptive interference cancellation technique has been used to the same purpose. Also proposed is a method to enhance atrial activity based on estimating the nonlinear transfer function (using a neural network) between two ECG leads. Additional approaches are based on a spatio-temporal processing of all the available leads, or a QRST complex cancellation technique based on blind source separation. Further, an approach based on multifractality parameters (generalized fractal dimensions) has been shown to be efficient in discriminating between ventricular tachycardia, ventricular fibrillation, and atrial fibrillation. An index of organization based on spectral estimation (ratio of the power in the harmonics to the total power) has been proposed to optimize defibrillation shock timing. Techniques for direct frequency analysis of AF from ECG without ventricular activity cancellation have been introduced, and the time-varying spectral characteristics of AF have recently been investigated. Further, methods have been introduced wherein, after QRST complex cancellation, the Wigner-Ville distribution is used to extract the instantaneous frequency and its trends. Finally, a different P-wave morphology, indicating atrial conduction defects, can be observed in patients with intermittent AF. Despite these recent advances, the problem of the clinicopathologic background of the arrhythmia and any relation to the electric morphology remained unsolved prior to the present invention.

### SUMMARY OF THE INVENTION

The present invention involves methods for detecting, describing, and analyzing cardiac arrhythmia in a patient, by detecting two or more atrial electrical segments in the patient, performing an autoregressive analysis on two or more atrial electrical segments, such that two or more test autoregressive coefficients (e.g., two, three, four, five, six, or ten coefficients) are determined for each of the atrial electrical segments, and comparing the test autoregressive coefficients with two or more standard autoregressive coefficients, wherein when the standard autoregressive coefficients are derived from one or more patients not suffering from or not at risk of suffering from a cardiac arrhythmia, a detectable difference in the test autoregressive coefficients as compared to the standard autoregressive coefficients indicates that the patient suffers from or is at risk of suffering from a cardiac arrhythmia, and wherein when standard autoregressive coefficients are derived from one or more patients suffering from or at risk of suffering from a cardiac arrhythmia, a detectable difference in the test autoregressive coefficients as compared to the standard autoregressive coefficients indicates that the patient does not suffer from or is not at risk of suffering from a cardiac arrhythmia. In embodiments of the invention, the cardiac arrhythmia is atrial fibrillation. The present invention allows for the identification of separate and distinct forms of atrial fibrillation, which was not possible previously. The invention therefore also provides for classifying the atrial fibrillation based on the detectable difference between the test autoregressive coefficients and the standard autoregressive coefficients.

As used herein, the atrial electrical segment include any interval between the S peak and the subsequent Q peak of an ECG. For example, the atrial electrical segment can include a rate-dependent interval between about 200 milliseconds before a Q peak and about 30 milliseconds before said Q peak. Alternatively, the atrial electrical segment can include a rate-dependent interval between about 300 milliseconds before a Q peak and about 30 milliseconds before said Q peak. Additionally, the atrial electrical segment can include a rate-dependent interval between about 500 milliseconds before a Q peak and about 30 milliseconds before said Q peak. In one embodiment, the present invention provides atrial electrical segments that are determined with about a 1 kHz sampling frequency.

The comparison between the test autoregressive coefficients and the standard autoregressive coefficients includes plotting the test autoregressive coefficients with the standard autoregressive coefficients in coefficient space. For example, when five test autoregressive coefficients are determined for each of said atrial electrical segments, the third test autoregressive coefficient and the fifth test autoregressive coefficient are plotted in coefficient space. The invention further provides for subjecting the plotted autoregressive coefficients to cluster analysis.

The invention includes classifying the atrial fibrillation as being consequent to any and all cardiac pathology or injury. By way of non-limiting example, the classified atrial fibrillation is selected from the group consisting of dilated cardiomyopathy, hypertrophic cardiomyopathy, rheumatismal valvular disease, pericarditis, idiopathic atrial fibrillation and focal atrial fibrillation.

The methods of the invention provide autoregressive analysis that account for a noise signal (e.g., white noise or wide noise).

The standard autoregressive coefficients of the invention are derived from one or more patients (e.g., one, two, three, four, five, six, ten, fifteen, twenty, fifty or more) with atrial fibrillation, e.g., atrial fibrillation caused by dilated cardiomyopathy, hypertrophic cardiomyopathy, rheumatismal valvular disease, pericarditis, ideopathic atrial fibrillation or focal atrial fibrillation. Alternatively, the standard autoregressive coefficients are derived from patients without detectable cardiac abnormalities. The standard autoregressive coefficients may constitute a database, either alone or in combination with other information.

In another aspect, the present invention provides a method for identifying a compound that modulates atrial fibrillation, including the steps of administering the compound to the patient, detecting two or more atrial electrical segments in the patient, performing an autoregressive analysis on each of the atrial electrical segments, such that two or more test autoregressive coefficients are determined for each of the atrial electrical segments, and comparing the test autoregressive coefficients with two or more standard autoregressive coefficients, wherein a detectable difference in the test autoregressive coefficients as compared to the standard autoregressive coefficients indicates that the candidate agent is a modulator of atrial fibrillation.

In another aspect, the present invention provides a method for identifying and classifying an atrial disorder in a patient suffering from or at risk of the atrial disorder, by detecting two or more atrial electrical segments in the patient, performing an autoregressive analysis on each of the atrial electrical segments, such that five or more test autoregressive coefficients are determined for each of the atrial electrical segments, providing two or more standard autoregressive coefficients, plotting two or more test autoregressive coefficients, and the standard autoregressive coefficients in coefficient space, and comparing the plotted test autoregressive coefficients with the plotted standard autoregressive coefficients, wherein an atrial disorder in the patient is identified and classified. The classified atrial disorder is dilated cardiomyopathy, hypertrophic cardiomyopathy, rheumatismal valvular disease, pericarditis, ideopathic atrial fibrillation, focal atrial fibrillation, or a similar cardiac disorder characterized, at least in part, by atrial fibrillation.

In another aspect, the present invention relates to a method for validating any model of atrial fibrillation, including the steps of generating two or more predicted atrial electrical segments, performing an autoregressive analysis on each of the predicted atrial electrical segments, such that five or more autoregressive coefficients are determined for each of the predicted atrial electrical segments, providing two or more test autoregressive coefficients (wherein the test autoregressive coefficients are generated by detecting two or more atrial electrical segments (e.g., such as detecting the atrial electrical segments in a mammalian patient), and performing an autoregressive analysis on each of the atrial electrical segments, such that five or more test autoregressive coefficients are determined for each of the test atrial electrical segment), plotting two or more autoregressive coefficients, in coefficient space, and comparing the plotted test autoregressive coefficients with the plotted autoregressive coefficients; whereby the model is validated thereby. By way of non-limiting example, a, model of atrial fibrillation is tested by generating two or more predicted atrial electrical segments, performing an autoregressive analysis on each of the predicted atrial electrical segments, such that five or more autoregressive coefficients are determined for each of the predicted atrial electrical segments, providing two or more test autoregressive coefficients (wherein the test autoregressive coefficients are generated by detecting two or more atrial electrical segments in a mammalian patient, and performing an autoregressive analysis on each of the atrial electrical segments, such that five or more test autoregressive coefficients are determined for each of the test atrial segment), plotting two or more of the autoregressive coefficients determined for each of the predicted atrial electrical segments and two or more test autoregressive coefficients, and comparing the plotted test autoregressive coefficients with the plotted autoregressive coefficients; thereby validating the model. The invention also provides the validated model generated by this method.

In another aspect, the present invention provides a model for atrial fibrillation, which includes a plurality of atrial electrical segments derived from two or more classified atrial disorders, which are subjected to an autoregressive analysis, such that two or more autoregressive coefficients are determined for each of the atrial electrical segments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 is an ECG trace demonstrating an exemplary atrial electrical segment of the present invention, measured from 200 milliseconds before one Q peak to 30 milliseconds before this Q peak. Action potential (in mV) is presented on the Y axis, and time (in milliseconds) is presented on the X axis.

FIG. 2 is a scatter plot illustrating one embodiment of the plotted test autoregressive coefficients of the invention. The third autoregressive coefficient is presented on the X axis, and the fifth autoregressive coefficient is presented on the Y axis.

### DETAILED DESCRIPTION OF THE INVENTION

The present invention provides new methods to diagnose the causal pathology of atrial fibrillations in a patient, such as a human patient with cardiovascular disease. Atrial fibrillation is an important clinical entity because of the increased risk of morbidity and mortality (1.5 to 1.9 fold in the Framingham study). The most frequent consequences of atrial fibrillation are hemodynamic function impairment (loss of atrial synchronized contraction, irregular and inadequately rapid ventricular rate), atriogenic thromboembolic events and tachycardia induced atrial and ventricular cardiomyopathy. Atrial fibrillation is usually associated with an underlying heart disease that produces elevated intra-atrial pressures and/or atrial dilatation (e.g., systolic or/and diastolic congestive heart failure, rheumatismal or non-rheumatismal valvular disease), atrial infiltration (e.g., amyloidosis, hemochomatosis, sarcoidosis), inflammation (e.g., myocarditis, pericarditis) or ectopic focus (e.g., premature atrial complexes). Atrial fibrillation most often appears clinically without identification of the underlying pathology (“lone AF”). As used herein, atrial fibrillation includes atrial flutter, including typical atrial flutter and atypical atrial flutter. The present invention is useful in the diagnosis of the causal patholgies underlying atrial flutter.

In physiologic conditions, the pacemaker function of the heart is ensured by the sino-atrial node. The cells of this node initiate regular waves of depolarization through atria and ventricles, with a rate of approximately 60-100 times per minute at rest in adult humans. Those cells can fire as fast as 180-200 times per minute at peak exercise. During AF and atrial flutter, the sino-atrial node loses its ability to act as the single unique pacemaker, and can be suppressed as atrial myocardium can fire at rates of 250-600 times per minute. The causal mechanism of AF in an individual patient is difficult to identify with the currently available tools, in part because atrial fibrillations are not able to be accurately classified.

Classification of atrial fibrillation based on the underlying pathology is an important diagnostic and therapeutic tool, due in great part to the complexity of managing atrial fibrillation. International guidelines for the management of patients with atrial fibrillation have been established. (See Fuster et al., Circ. 104:2118 (2001)). With the goal of treatment being the prevention of the deleterious effects induced by AF, two approaches are employed: the restoration of the sinusal rhythm by pharmacological or electrical cardioversion, and acceptation of AF with pharmacological prevention of complications. Cardioversion, either with drugs or by electrical means, is not always successful, and even when achieved, sinusal rhythm maintenance is observed in only 42% and 27% of patients after 1 and 4 years, respectively. (See Van Gelder et al., Arch. Intern. Med. 156:2585 (1996)). A further complication is that cardioversion is even less efficient with patients presenting a chronic AF exceeding one year, due to structural (e.g., fibrotic) and electric remodeling of the atrium. For chronic AF, pharmacological control of the atrio-ventricular conduction is attempted, in order to maintain acceptable hemodynamics and prevent tachycardia-induced cardiomyopathy. In most situations thromboembolic prophylaxis is mandatory. This approach is not optimal because of the increased risk of side effects, including bradycardia or haemorrhage. Other recent proposed approaches include surgical intervention, pacing AF, internal cardioversion, linear ablation and pulmonary vein isolation. A deficiency of these procedures and therapies is that they are based on empirical observations, and with the exception of pulmonay vein isolation, are not specific treatments and do not take in account the underlying pathology responsible of the arrhythmia.

Several descriptive classifications based on any mode of appearance or etiology, duration, substrate, efficiency of treatment have been proposed, yet none are satisfactory in the absence of specific therapeutic implications.

The present invention provides methods to detect cardiac arrhythmias (e.g., atrial fibrillation) in patients, such as humans, and to classify these atrial fibrillations based on newly defined ECG parameters.

In one aspect, the present invention provides a method for detecting cardiac arrhythmia in a patient, the method including the steps of detecting two or more atrial electrical segments in the patient, performing an autoregressive analysis on each of the atrial electrical segments, such that two or more test autoregressive coefficients are determined for each of the atrial electrical segments, and comparing the test autoregressive coefficients with two or more standard autoregressive coefficients, where a detectable difference in the test autoregressive coefficients as compared to the standard autoregressive coefficients indicates that the patient suffers from or is at risk of a cardiac arrhythmia, and indicates the classification of the atrial fibrillation.

Data regarding a patient's cardiac situation is determined non-invasively, such as with an electrocardiogram. Typically, an external 12-lead ECG is used, but any device capable of detecting the electrical activity of the heart can be employed. ECG recording is performed for a sufficient period of time at a sampling frequency sufficient to provide a statistically significant number of atrial electrical segments. Generally, ECG recording occurs for about 10 minutes, but can be of longer or shorter duration. The ECG sampling frequency is about 1 kHz. Alternatively, the sampling frequency is about 100 Hz, 250 Hz, 500 Hz, 750 Hz, 900 Hz, 1.1 kHz, 1.25 kHz, 1.5 kHz, or more.

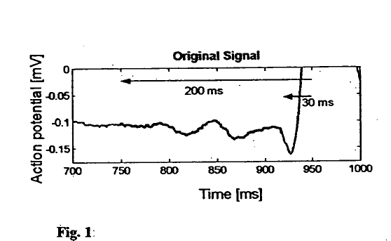

As used herein, the term “atrial electrical segment” includes an ECG reading between two consecutive Q peaks. Generally, atrial electrical segments are extracted from the ECG readings by first detecting the R and Q peaks. The R and Q peaks can be detected using peak detection methods known in the art, such as methods based on thresholding and product of derivatives analysis. A specific Q peak is selected and a window of time preceding this specific Q peak is established. The duration of the atrial electrical segment can be as great as the time between the end of an R peak and before the Q peak immediately subsequent thereto, but is generally of shorter duration, such as between the end of an S peak and before the Q peak immediately subsequent thereto. For example, the atrial electrical segment is 170 milliseconds (ms) in duration. In other embodiments, the atrial electrical segment is 20, 50, 100, 150, 200, 250, 300, 350, 400, 500, 600 or more ms in duration. In an embodiment of the invention, the atrial electrical segment includes that portion of the ECG reading that is 170 ms in duration, beginning 200 ms before a Q peak and ending 30 ms before that Q peak. See, e.g., FIG. 1. In other embodiments, the atrial electrical segment includes a portion that begins at 100, 150, 200, 300, 400, 500 or more ms before a Q peak and ends 5, 10, 15, 20, 30, 50, 100 or more ms before that Q peak. Thus, an ECG recording 10 minutes in duration with a sampling frequency of 1 kHz provides about 700-800 atrial electrical segments.

Each atrial electrical segment was then subjected to autoregressive analysis to derive autoregressive (AR) coefficients for each atrial electrical segment.

Autoregressive Analysis

The present invention provides autoregressive analysis using autoregressive models to define the relationship between successive atrial electrical segments. One model is defined-as follows:

$$x ( n ) + {\sum }_{i = 1}^{P} {a}_{i} x ( n - i ) + e ( n )$$

Where x(n) is the sample at time point n of the signal x. e is the white noise in the model, i.e., generally a Gaussian, zero-mean signal with values that are not correlated with signal x. The order p is determined statistically, and can be one, two, three, four, five, six or more. The order can also be determined using the Akaike information criterion. The autoregressive coefficient aᵢ is determined for each atrial electrical segment. In embodiments of the invention, one, two, three, four, five, six or more autoregressive coefficients are determined for each atrial electrical segment. While aᵢx(n-i) represents the portion of x(n) that is capable of being inferred from surrounding atrial electrical segments (e.g., preceding atrial electrical segments of succeeding atrial electrical segments), e(n) represents the portion of x(n) that is cannot be inferred from surrounding atrial electrical segments, and is thus termed the innovation. The autoregressive analysis accounts for the white noise signal that is present, if at all, in the atrial electrical segment.

The AR coefficients are determined using Burg method (based on Burg's algorithm) or a least squares method (e.g., least squares based upon the Yule-Walker equations).

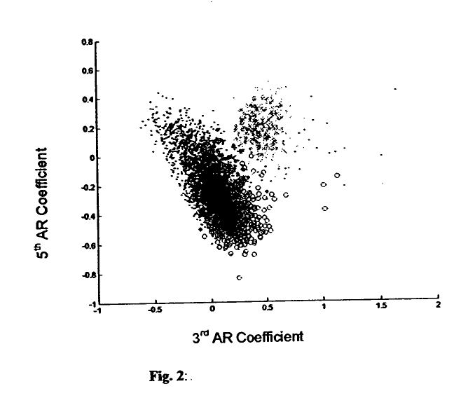

The AR coefficients for a patient's atrial electrical segments are plotted in coefficient space. When two or more patients are compared with each other, the AR coefficients for every patient are plotted together. The AR coefficients to be plotted are selected based on either their variance over time for a single patient, or their variance among patients, or both. By way of non-limiting example the 5^{th} AR coefficient is plotted on the y-axis and the 3^{rd} AR coefficient is plotted on the x-axis. See, e.g., FIG. 2. A patient's AR coefficients can also be plotted along with one or more standard AR coefficients. A standard AR coefficient is derived from one or more subjects (e.g., one, two, three, four, five, six, ten, fifteen, twenty, fifty or more) that are known to have a specific classification of an atrial fibrillation e.g., atrial fibrillation caused by dilated cardiomyopathy, hypertrophic cardiomyopathy, rheumatismal valvular disease, pericarditis, or focal atrial fibrillation. Alternatively, a standard AR coefficient is derived from one or more subjects known to have a normal sinus rhythm. One or more standard AR coefficients may be included in a database of known AR or known unaffected individuals. The database may contain other information relating to AF classification, such as patient history or other medical tests performed.

Classification of the atrial fibrillation of a given patient or group of patients is performed by visual analysis of the plotted AR coefficients. Alternatively, the AR coefficient values are subjected to a mathematical cluster analysis, including hierarchical methods (e.g., single linkage, average linkage (weighted and unweighted), centroid, median and complete linkage) and non-hierarchical methods (e.g., the K-means clustering algorithm, adaptive K-means, K-medoids, and fuzzy clustering).

Detection of a cardiac arrhythmia in a patient includes comparing the test AR coefficients with two or more standard autoregressive coefficients. A detectable difference between the two types of coefficients indicates that the patient suffers from or is at risk of a cardiac arrhtymia such as atrial fibrillation. The detectable difference can be identified by visual analysis of the plotted AR coefficients, by mathematical cluster analysis, or other detection means known in the art.

The present invention also provides methods for identifying compounds that modulate atrial fibrillation. A test compound is administered to a patient. Routes of administration are known in the art, and include parenteral and non-parenteral routes of administration. As used herein, “patient” includes any mammal whose cardiac electrical is capable of being monitored, such as by an ECG. For the purposes of identifying compounds that modulate atrial fibrillation, preferred mammals include primates, rodents (e.g., mice, rats, guinea pigs, hamsters, etc.), dogs, rabbits, pigs and cats. In embodiments of the invention, modulation of atrial fibrillation is a decrease or inhibition in atrial fibrillation. Alternatively, compounds that modulate atrial fibrillation initiate or increase atrial fibrillation.

### EXAMPLES

The invention will be further described in the following examples, which do not limit the scope of the invention described in the claims.

### Example 1

### Atrial Fibrillation Classifications Derived from Autoregressive Analysis of Patient Electrocardiograms

The present invention provides methods to detect and classify (e.g., to describe) patterns of cardiac arrhythmias (e.g., atrial fibrillation) in patients, such as humans. Electrocardiograms from five human subjects, two having sino-atrial rhythm (normal sinus rhythm) and three having atrial fibrillation were analyzed.

Methods. Each subject was connected to an external 12-lead ECG. Recording was performed for 1 minutes at a 1 kHz sampling frequency. Atrial electrical segments were extracted from the ECG readings by first detecting the R and then Q peaks using peak detection methods based on thresholding and product of derivatives analysis, then defining an atrial electrical segment as a 170 millisecond (ms) window beginning 200 ms before a Q peak and ending 30 ms before that Q peak. This definition provides atrial electrical segments containing 170 sample points that are free of Q, R, and S peaks, as shown in FIG. 1. The mean value of each atrial electrical segment Was then subtracted. Thus, for each patient, one ECG recording contains about 700-800 atrial electrical segments.

Each atrial electrical segment was then subjected to autoregressive analysis to derive five autoregressive (AR) coefficients for each atrial electrical segment. The AR coefficients were determined with the Burg method, which estimates autoregressive parameters by minimizing the forward and backward prediction in the least-square sense while conforming to the Levinson-Durbin recursion. The third and fifth AR coefficients derived for each atrial electrical segment of all five patients were plotted in coefficient space by plotting the 5^{th} AR coefficient on the y-axis and the 3^{rd} AR coefficient on the x-axis.

Results. The resulting plot is shown in FIG. 2. The AR coefficients for the two patients with sino-atrial rhythm are shown as open circle marks (S-A Patient 1) and small closed black circle marks (S-A Patient 2). The AR coefficients for the three patients with atrial fibrillation are shown as small closed grey circle marks (AF Patient 1), grey cross marks.(AF Patient 2), and black star marks (AF Patient 3). Visual analysis of the coefficient plot illustrates that several distinct clusters exist. The two patients with sino-atrial rhythm are very close together and are in the same cluster. The AF Patient 1 cluster is in close proximity to the sino-atrial rhythm cluster but can still be distinguished from it. The AF Patient 2 cluster is well-separated from the other clusters and is classified as a distinct type of atrial fibrillation. The cluster of AF Patient 3 overlaps with the sino-atrial rhythm cluster and the AF Patient 1 cluster. Thus, AF Patient 3 can be classified as having a fibrillation type with electrical features distinct from the other forms of atrial fibrillation. Mathematical cluster analysis provides quantitative distinctions among the classified atrial fibrillation patients. Each of these patients had a distinct underlying mechanism causing the AF, which was diagnosed using the classification methods of the present invention.

### Example 2

### Creation and Validation of Models of Cardiac Electrical Activity

In vivo measurement of cardiac electrical activity is a non-trivial undertaking, and in vitro use small tissue preparations or isolated cells, making the use of computer-derived models attractive. Experiments performed on explanted hearts have always strong limitations since a complete access to the whole heart is usually not achieved, therefore the electrical activity recording is restricted to a limited area of the heart surface. Further, no transmural information is generally available and the spatial and temporal range of available information is reduced because of the limitation of the acquisition devices (e.g., the number of channels of analogic-to-digital converters). While these limitations do not greatly restrain an understanding of a normal beating heart, they are detrimental to a greater understanding of abnormal cardiac propagations (e.g., arrhythmias and fibrillation), which have for many years been recognized to result from various complex spatio-temporal mechanisms intimately coupled to particular defect of the myocardial substrate or structure. Therefore, building a realistic computer model of the heart (or, at the very least to one of its subparts) will dramatically refine our understanding of arrhythmia processes, through the easy and complete access to data, and through the ability to carefully control each parameter of the model, thus allowing the uncoupling of closely linked phenomena.

An accurate model of electrical activity must incorporate three basic descriptions of current flows: one for the intracellular domain, one for the extracellular one and one for the ion exchanges through the cell membrane. The transmembrane current model determines how the action potential (voltage pulse across the membrane generated as the response to an electrical stimulation) is approximated in the model. Historically, very simple models have first been proposed: cellular automata based-formalism exhibiting very basic excitation properties based on a discrete state description. The next class of models widely used is the two-variable formulation of the FitzhHugbNagumo family. These models approximate the excitation process through two arbitrary currents, described by first order non-linear differential equations, one for the fast depolarization process and the second one for the slower repolarization phase. While providing a much higher level of refinement than cellular-automaton approaches, these models have also substantial limitations if complex spatio-temporal interactions arise (like the those encountered during arrhythmias). Finally, the third family is the one of ionic models. These are based on extended patch-clamped measurements of the electrical exchanges through the various ion channels involved in transmembrane currents. The measured behavior of the channels is then reproduced by non-linear curve fitting, resulting in a set of non-linear first order differential equations, each equation describing one particular transmembrane current.

The genesis of ionic models was the work of Hodgkin and Huxley on the excitable properties of the giant squid axon. The formalism developed in this work acted as a basis for the simplified two-variable FitzhHugh-Nagumo family model and to models specifically targeted towards particular cardiac cell types. The Purkinje fibers were investigated first, followed by the mammal myocardiac cell model of Beeler and Reuter and its reformulation by Luo and Rudy. Other ventricular ionic models were developed by DiFrancesco and Nobel to take into account ionic pumps, ion buffering and concentration changes, features that were incorporated by Luo and Rudy in revised versions of their formulation. Most efforts have been directed towards ventricular cells, and only a few models have been developed for atrial cells. The first targets were the sino-atrial cells responsible for pacemaker activity.

Recent models of healthy atrial cells by Nygren et al. and Courtemanche et al. incorporate ionic pumps, ion buffering and concentration changes. However, the implementation of these models in spatially extended models (as opposed to single isolated cells) is not common yet, although Harrild and Henriquez have used a Nygren formulation in their model of healthy atrial propagation. Until the present invention, the ability of a model to adequately reproduce atrial arrhythmia for several seconds has not been established. The present invention-provides membrane models that are focused upon the role(s) of the atria in cardiac fitness.

Accurate modeling of current flow is an important function of the cardiac model of the present invention. Current flow in the intracellular and extracellular domains describes how the electrical excitation is transmitted from one cell to another. Both of these domains exhibit marked discrete structures. In the case of the intracellular domain, current flow is significantly influenced by cell compartmentalization (i.e., the alternation of low resistive cytoplasmic compartments with high resistive gap junctions). The extracellular interstitial space is also structured by cell-to-cell organization. Nevertheless, this discrete reality has usually been discarded or simplified when building numerical models of both domains: the myocardium is usually considered as a continuous medium. It was believed that such a simplification was required to lower the computational requirements of coupled cell computations.

The simultaneous computation of intracellular and extracellular flows in a bidomain model leads to complex discrete formulations requiring complex solving schemes that are known to have a low efficiency. Therefore, a single propagation medium is generally assumed in all models, which takes into account large portions of the myocardium, and is dedicated to the simulation of long runs of cardiac propagation. This simplification is achieved either by considering a compound domain of the intracellular and extracellular media having equal anisotropy ratio, or by assuming an isopotential extracellular medium and computing only the intracellular space. In an embodiment of the present invention, a monodomain formulation is provided.

While the efforts at cardiac modeling have historically been directed towards ventricular models and ventricular arrhythmias, progress has been made in establishing models that include the atria. The work of Harrild and Heniquez describes a complete three-dimensional atrial structure and the Nygren et al. ionic model. However, this model is not suitable for studying arrhythmic propagations in part because of its computational requirements. Computational models specifically dedicated towards atrial arrhythmia simulations were developed using simplified geometrical approaches, with the assumption of atrial walls thin enough to be considered as a single layer of tissue from an electrophysiological viewpoint. While a marked trend towards realistic human atrial anatomy that overcomes the limits of simplified geometries has progressed recently, the monolayer assumption continues to be used because of computational requirements.

In whole atrial arrhythmic models, the tissue is usually assumed to be isotropic and homogeneous. One exception to this rule is a specific particular fiber model, but this model imposes non-realistic limitations to atrial anatomy. The isotropic and homogeneous assumption appears now to be one of the major limitations of the existing models, since it is clear that structural, as well as electrophysiological heterogeneities are associated with arrhythmia initiation and perpetuation, specifically in the case of diseased and aged fibrillating atria. The preliminary models incorporating heterogeneous islands in two-dimensional square sheets of tissue developed recently confirmed the need to overcome the isotropic and homogeneous assumption, a need which was even more acute in regard to whole organ models.

The present invention provides a pseudo three-dimensional model of human atria that provides the main anatomic features including veins and valve openings. The present model is an advancement over prior versions, which were monolayers of tissue folded over a simplified atrial geometry having realistic dimensions, based on the Beeler-Reuter formulation. The prior model was capable of simulating atrial flutter and atrial fibrillation, and stressed the importance of the main anatomic obstacles on AF dynamics Preliminary simulations of radio-frequency ablations have also been performed and have appeared to be in good agreement with clinical results. To our knowledge, this model was the first one to be directed specifically towards the reproduction of AF arrhythmias. It has demonstrated the feasibility of the approach.

Major enhancements have been brought to this model. Its current state is now based on a real, measured human atrial anatomy (obtained in vivo through MRI techniques). Specific numerical techniques have been developed to compute propagation in the monolayer tissue described by a triangular mesh. The membrane activation is computed using an “atrialized” version of the Luo-Rudy dynamics, deeply modified, that now provides for the simulation of healthy tissue and electrically remodeled cells. In an embodiment of the present invention, AF is initiated through high frequency stimulation in the right atrium, using protocols similar to those employed in electrophysiological experiments. Single runs of up to 4 minutes of AF have been simulated (requiring approx. 3 weeks of computation on a single Pentium IV 1800 MHz processor, with 100,000 spatial nodes). The present invention includes single runs of 5 minutes, 10 minutes, 20 minutes, 30 minutes, 45 minutes, 1 hour, or greater than one hour. Further, the present invention provides a model having greater than 150000, 200000, 500000, 1 million or more spatial nodes.

The model of the present invention provides for the accurate reproduction of local electrograms. As previously discussed, the present invention provides for the identification and classification of the underlying atrial disorder in a patient. Once atrial fibrillations are classified on the basis of the underlying atrial disorder using ECG, these classified atrial fibrillations are simulated in the cardiac model of the present invention. Specific targeted atrial disorders include dilated cardiomyopathy, hypertrophic cardiomyopathy, rheumatismal valvular disease, pencarditis and ectopic foci.

In one embodiment of the present invention, the cardiac model is created and validated as follows.

Task 1. External ECG and Intracardiac Electrogram Measurement during AF Episodes from a Subset of Patients Belonging to Target Groups of AF.

The patients are selected from the currently treated people in the cardiology service of CHUV. Selection is based on their known clinical history. For further analysis purposes, multiple pathologies leading to an unclear classification among the five target groups is avoided. Intracardiac recordings consist of AF runs of several seconds (e.g., about 10 seconds), recorded in multiple locations of the right atrium. External 12-lead ECG are also performed accordingly. Several ECG recordings are performed at 2-3 weeks intervals, first to assess infra-patient AF stability through time, and second, for inter-patient classification. Intracardiac measurements are expected to provide a “closer” view on AF, with recordings restricted to the vicinity of the electrodes only, while 12-lead ECG should provide a “wider” view on AF, with the major drawback of atrial signals being immersed into the ventricular activity. For analysis purposes (See Task 2, below), a minimal number of 3 patients (e.g., 3, 4, 5, 6, 7, 8, 9, 10 or more) patients for each group is targeted. For validation purposes (See Task 6, below), 1 or more patients (e.g., 1, 2, 3, 4, 5, 6, 7, 8, 9, 10 or more) per group are provided.

### Task 2: Analysis and Classification of AF Signals

AF is a complex, time-varying spatio-temporal process which can be observed with the help of electrograms, or more easily with ECG. AF signals can be classified as described in Example 1. The invention provides for the combination of available signals (e.g., electrograms and ECG leads), which is useful to cancel interference, reduce noise, to remove redundancies, extract the most representative components, and quantify the relationships between these signals. Also, information is typically present in the time evolution of the phenomenon, i.e., in the rate of change of the estimated parameters. In embodiments of the present invention, one or more of the following approaches are employed to classify or order AF signals:

Separation techniques. As used herein, “separation techniques” include principal component analysis (PCA), independent component analysis (ICA), and blind source separation (BSS), with reference to convolutive mixtures. This approach allows for the simultaneous management: of interference cancellation and information-bearing source extraction.

State-space techniques. As used herein, “state-space techniques” include techniques inspired by or derived from chaos theory. Although chaos theory in itself has only a limited impact on applied problems, the development of estimation techniques for invariants such as dimension and Lyapunov exponents and for process complexity quantification has opened new avenues for the characterization of biomedical phenomena. Other approaches include the use of multivariate AR models.

Time-frequency techniques. The time evolution of AF originating from various substrates is determined, in order to extract possible time evolution patterns indicative, of these various substrates.

Generalized coherence techniques. As classical coherence is only sensitive to linear, symmetric, and stationary interdependencies between signals, the present invention provides methods that resolve nonlinear and/or non-stationary situations as well as asymmetric interdependencies, in order to resolve the spatiotemporal evolution of AF.

ECG-based substrate determination is a preferred embodiment. Further, due to the “global” nature of ECG (i.e., an ECG is the result of the electrical activity of the whole cardiac muscle), the ECG is able to yield more information about the state of the tissue than local electrogram signals.

### Task 3: AF Model: External ECG Modeling.

The present invention provides the ability to reproduce external ECG signals and as demonstrated above, intracardiac electrogram simulation has been demonstrated to adequately reproduce experimental results. The local electrogram computation relies on simplified torso properties (assumed to be an unbounded homogeneous medium). However, this hypothesis is not valid once a far field electrocardiogram at the body surface is simulated. Therefore, the limited dimensions of the torso, and the boundary effects at the abrupt discontinuity between the conductive tissue and the surrounding nonconductive air, are known to play a major role and must be considered in a comprehensive cardiac model. The development of a simplified torso model is therefore mandatory, together with the mathematical methods allowing fast estimations of the ECG signal at a limited number of points of the surface. A homogeneous ellipsoidal cylinder is employed, with the ability to further include high resistive obstacles like the lungs (again based on simplified geometries, i.e., ellipsoids and/or cylinders). To implement this task, the present invention provides for the implementation of a boundary-element method. Model validation is achieved with determination of non-AF signals and the classification of AF ECG signals, as described in Task 1 and Example 1.

### Task 4: AF Model: Pathological Modifications

The present invention provides a precise determination of the manner in which the electrical and structural properties of the atrial myocardium are affected in particular pathologies. This determination is then integrated into the model. Mathematical methods are used that control for a degree of heterogeneity within the classified pathologies. These methods require the ability to adequately compute electrical propagation in an heterogeneous and anisotropic medium, as well as the ability to control and modify these functions. Therefore, also provided are methods to develop the mandatory user-interface tools to manipulate these functions in three-dimensional space.

Upon integration of the structural and electrophysiological modifications specific of each classified AF etiology, the model is validated against the published literature and clinical evidence to confirm predicted AF initiation, perpetuation mechanisms and, optionally, spontaneous termination.

Task 5: Testing AF Classification on the Computer Model.

The AF model is used to simulate intracardiac electrograms and external ECG. The classification techniques developed in, e.g., Task 2, are applied to these synthetic signals. This phase is provides one or more iterations with Task 4, until AF models are validated (i.e., reach a satisfactory level of agreement with the experimental findings). Further, the new AF classification described above, which is based on the analysis of the biological signal, will also benefit from the computer models, since the stable control of the state of the myocardium achieved in a computer model is advantageous in testing, for example classification hypotheses.

Task 6: Validation of the New Classification Techniques.

The tested AF classification is then blindly applied to a set of external ECG and intracardiac electrograms obtained by the methods described in Task 1, e.g., patients suffering from AF having an unknown etiology. The results obtained from the computer model are then compared to known patient history. The model allows the determination of whether, based on electrical activity recording, different mhyocardial substrates lead to significantly different AF once AF is established and sustained for a period of time. Further, the model indicates the amount of data required to perform the classification, and indicates, for an individual patient whether data derived from external ECG measurement is sufficient to generate a valid classification, or if intracardiac data must be included.

### OTHER EMBODIMENTS

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for detecting cardiac arrhythmia in a patient, comprising the steps of:
(i) detecting two or more atrial electrical segments in said patient;
(ii) performing an autoregressive analysis on each of said atrial electrical segments, such that two or more test autoregressive coefficients are determined for each of said atrial electrical segments; and
(iii). comparing said test autoregressive coefficients with two or more standard autoregressive coefficients;
wherein, when said standard autoregressive coefficients are derived from one or more patients not suffering from or not at risk of suffering from a cardiac arrhythmia, a detectable difference in said test autoregressive coefficients as compared to said standard autoregressive coefficients indicates that said patient suffers from or is at risk of suffering from a cardiac arrhythmia, and wherein, when said standard autoregressive coefficients are derived from one or more patients suffering from or at risk of suffering from a cardiac arrhythmia, a detectable difference in said test autoregressive coefficients as compared to said standard autoregressive coefficients indicates that said patient does not suffer from or is not at risk of suffering from a cardiac arrhythmia.

2. The method of claim 1, wherein said cardiac arrhythmia is atrial fibrillation.

3. The method of claim 2, further comprising classifying said atrial fibrillation based on said detectable difference.

4. The method of claim 2, wherein said comparing comprises plotting said test autoregressive coefficients with said standard autoregressive coefficients in coefficient space.

5. The method of claim 4, wherein five or more test autoregressive coefficients are determined for each of said atrial electrical segments.

6. The method of claim 4, wherein five test autoregressive coefficients are determined for each of said atrial electrical segments, wherein two or more test autoregressive coefficients are plotted in coefficient space.

7. The method of claim 6, wherein said plotted autoregressive coefficients are subjected to cluster analysis.

8. The method of claim 3, wherein said classified atrial fibrillation is selected from the group consisting of dilated cardiomyopathy, hypertrophic cardiomyopathy, rheumatismal valvular disease, pericarditis, ideopathic atrial fibrillation, and focal atrial fibrillation.

9. The method of claim 1, where said autoregressive analysis accounts for a noise signal.

10. The method of claim 1, wherein said atrial electrical segment comprises an interval between the S peak and the subsequent Q peak.

11. The method of claim 1, wherein atrial electrical segment comprises an interval between about 500 milliseconds before a Q peak about 30 milliseconds before said Q peak.

12. The method of claim 1, wherein atrial electrical segment comprises an interval between about 300 milliseconds before a Q peak about 30 milliseconds before said Q peak.

13. The method of claim 1, wherein atrial electrical segment comprises an interval between about 200 milliseconds before a Q peak about 30 milliseconds before said Q peak.

14. The method of claim 1, wherein said atrial electrical segments are detected with about a 1 kHz sampling frequency.

15. The method of claim 1, wherein said standard autoregressive coefficients are derived from one or more patients without atrial fibrillation.

16. The method of claim 1, wherein said standard autoregressive coefficients are derived from one or more patients with atrial fibrillation, wherein said patients have atrial fibrillation caused by dilated cardiomyopathy, hypertrophic cardiomyopathy, rheumatismal valvular disease, pericarditis, or focal atrial fibrillation.

17. The method of claim 1, wherein said standard autoregressive coefficients comprise a database.

18. A method for identifying a compound that modulates atrial fibrillation, comprising the steps of:
(i) administering said compound to said patient;
(ii) detecting two or more atrial electrical segments in said patient;
(iii) performing an autoregressive analysis on each of said atrial electrical segments, such that two or more test autoregressive coefficients are determined for each of said atrial electrical segments; and
(iv) comparing said test autoregressive coefficients with two or more standard autoregressive coefficients;
wherein a detectable difference in said test autoregressive coefficients as compared to said standard autoregressive coefficients indicates that said candidate agent is a modulator of atrial fibrillation.

19. A method for identifying and classifying an atrial disorder in a patient suffering from or at risk of suffering from said atrial disorder, wherein said atrial disorder results in atrial fibrillation, comprising the steps of:
(i) detecting two or more atrial electrical segments in said patient;
(ii) performing an autoregressive analysis on each of said atrial electrical segments, such that five or more test autoregressive coefficients are determined for each of said atrial electrical segments;
(iii) providing two or more standard autoregressive coefficients, wherein said standard autoregressive coefficients are derived from patients suffering from said atrial disorder;
(iv) plotting said two or more standard autoregressive coefficients and said standard autoregressive coefficients in coefficient space;
(v) comparing said plotted test autoregressive coefficients with said plotted standard autoregressive coefficients;
wherein a detectable similarity in said test autoregressive coefficients as compared to said standard autoregressive coefficients indicates that said patient suffers from or is at risk of suffering from said atrial disorder, and wherein a detectable difference in said test autoregressive coefficients as compared to said standard autoregressive coefficients indicates that said patient does not suffer from is not at risk of suffering from said atrial disorder.

20. The method of claim 19, wherein said classified atrial disorder is selected from the group consisting of dilated cardiomyopathy, hypertrophic cardiomyopathy, rheumatismal valvular disease, pericarditis, ideopathic atrial fibrillation and focal atrial fibrillation.

21. A method for validating a model of atrial fibrillation, comprising the steps of:
(i) generating two or more predicted atrial electrical segments from said model;
(ii) performing an autoregressive analysis on each of said predicted atrial electrical segments, such that, five or more autoregressive coefficients are determined for each of said predicted atrial electrical segments;
(iii) providing two or more test autoregressive coefficients, wherein said test autoregressive coefficients are generated by:
a. detecting two or more atrial electrical segments in a mammalian patient suffering from atrial fibrillation; and
b. performing an autoregressive analysis on each of said atrial electrical segments, such that five or more test autoregressive coefficients are determined for each of said atrial electrical segments.
(iv) plotting two or more of said autoregressive coefficients and two or more of said test autoregressive coefficients in coefficient space; and
(v) comparing said plotted test autoregressive coefficients with said plotted predicted autoregressive coefficients;
wherein a detectable similarity in said plotted test autoregressive coefficients as compared to said predicted autoregressive coefficients indicates that said model is valid for atrial fibrillation.

22. A validated model obtainable according to the method of claim 21.

23. A model for atrial fibrillation, comprising a plurality of atrial electrical segments, said plurality derived from two or more classified atrial disorders, wherein said plurality of atrial electrical segments is subjected to an autoregressive analysis on each of said atrial electrical segments, such that two or more autoregressive coefficients are determined for each of said atrial electrical segments.

24. The model of claim 23, wherein said classified atrial disorders are selected from the group consisting of dilated cardiomyopathy, hypertrophic cardiomyopathy, rheumatismal valvular disease, pericarditis, ideopathic atrial fibrillation, and focal atrial fibrillation.

25. The model of claim 24, wherein said atrial electrical segments are ordered using separation techniques, state-space techniques, time-frequency techniques, or generalized choherence techniques.
